(19) United States
(12) Patent Application Publication
(10) Pub. No.: US 2010/0216162 A1
(43) Pub. Date: Aug. 26, 2010

(54) Immunological Method, Test Kit and Device for the Determination of the Analyte Content of a Sample

(75) Inventors: Marcus Meyr, Schwabmunchen (DE); Peter Schneider, Stadtbergen (DE); Friedrich Frank, Oberostendorf (DE)

Correspondence Address: SALIWANCHIK LLOYD & SALIWANCHIK;A PROFESSIONAL ASSOCIATION, PO Box 142950, GAINESVILLE, FL, 32614, US

(73) Assignees: MINITÜLL-UND LABORTECHNIK GMBH & CO. KG, Tiefenbach (DE); FRIMTEC GMBH, Oberostendorf (DE); SENSION, BIOLOGISCHE DETEKTIONS- UND SCHNELLTESTS, Augsburg (DE)

(21) Appl. No.: 12/601,648

(22) PCT Filed: May 30, 2008
(86) PCT No.: PCT/EP2008/004322

§ 371 Date: May 10, 2010

(30) Foreign Application Priority Data
May 30, 2007 (DE) 20 2007 007 675.4
Jan. 30, 2008 (DE) 10 2008 006 806.3

(51) Int. Cl. G01N 33/53; C12M 1/34

(52) U.S. Cl. 435/7.9; 435/287.2

(57) ABSTRACT

The present invention relates to immunological methods for the determination of the analyte content in samples as well as to test kits and devices for performing the methods of the invention. In particular, the present invention relates to the analysis of samples of breeding animals, such as raw milk and serum, for an assessment of desired characteristics of the animal(s) tested, such as pregnancy, optimum point of insemination or diseases, wherein said analysis is easy to handle, time efficient and can be carried out on-site.

The present invention relates to immunological methods for the determination of the analyte content in samples as well as to test kits and devices for performing the methods of the invention. In particular, the present invention relates to the analysis of samples of breeding animals, such as raw milk and serum, for an assessment of desired characteristics of the animal(s) tested, such as pregnancy, optimum point of insemination or diseases, wherein said analysis is easy to handle, time efficient and can be carried out on-site.

### BACKGROUND OF THE INVENTION

The determination of substances or analytes, respectively, in samples of breeding animals, for instance progesterone in milk, whole blood or serum, is of big importance in the fields of agriculture and animal breeding, especially for reproduction management purposes, in particular to reliably determine the optimum point in time for insemination. As a successful insemination only can be performed within a limited time frame, fast and uncomplicated on-site procedures are required.

DE 197 05 163 C1 and EP 0 671 006 B1 describe various ELISA-based rapid tests. Furthermore DE 101 19 696 B4 describes a dosage device and a dosage procedure, as it can be also partially applied with the invention presented here.

It is an object of the invention to provide an easy and accurate procedure, an easy to handle test kit and test device for the on-site determination of the substance content of samples.

### SUMMARY OF THE INVENTION

According to the present invention this object is solved by providing immunological methods for the determination of the analyte content in a sample.

In one embodiment, a method for the determination of the analyte content in a sample according to the present invention preferably comprises the following steps

(a) providing at least one container, wherein the inner surface of said container is at least partially coated with an anti-analyte antibody,

(b) adding the sample and an analyte-enzyme conjugate to the at least one container,

(c) incubating the sample and the analyte-enzyme conjugate in the at least one container,

(d) removing the mixture of sample and analyte-enzyme conjugate from the at least one container,

(e) adding a precipitating enzyme substrate to the at least one container, wherein the substrate preferably comprises a precipitating chromogen, and

(f) detecting the colour value of the inner surface of the at least one container, wherein the colour value is the shade of colour, the colour saturation, the white shade value and/or the grey scale level,

(g) optional, converting the colour value detected in step (f) into a value of the analyte content of the sample.

In another embodiment, a method for the determination of the analyte content in a sample according to the present invention preferably comprises the following steps

(a) providing at least one container, wherein the inner surface of said container is at least partially coated with a primary anti-analyte antibody,

(b) adding the sample to the at least one container,

(c) incubating the sample in the at least one container and removing it therefrom,

(d) adding a secondary antibody conjugated with enzyme to the at least one container, incubating the secondary antibody in the at least one container and removing it therefrom,

(e) adding a precipitating enzyme substrate to the at least one container, wherein the substrate preferably comprises a precipitating chromogen, and

(f) detecting the colour value of the inner surface of the at least one container, wherein the colour value is the shade of colour, the colour saturation, the white shade value and/or the grey scale level,

(g) optional, converting the colour value detected in step (f) into a value of the analyte content of the sample.

According to the present invention this object is furthermore solved by providing a test kit for the determination of the analyte content in a sample. The test kit is preferably for performing the methods of the invention and/or for use in the device of the invention.

According to the present invention this object is furthermore solved by providing a device for the determination of the analyte content in a sample. The device is preferably for performing the methods of the invention and/or for the use with the test kit of the invention.

### DESCRIPTION OF THE PREFERRED EMBODIMENTS OF THE INVENTION

Before the present invention is described in more detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art. For the purpose of the present invention, all references cited herein are incorporated by reference in their entireties.

Immunological Detection Methods

As outlined above, the present invention provides methods for the determination of the analyte content in a sample.

The sample for the methods, test kits and devices of the present invention is preferably an untreated sample, wherein “untreated” refers to not processed (i.e. without any physical and/or chemical treatment) and/or undiluted. A preferred untreated sample in the present invention is raw milk, i.e. foremilk, mainmilk or hindmilk, which is directly used in the present invention, i.e. without the need of dilution or any physical and/or chemical treatment.

The sample is preferably selected from bodily fluids, such as raw milk, urine, saliva, whole blood, blood plasma or blood serum.

The sample is furthermore preferably derived from a mammal or a bird, such as human, cow, pig, horse, dog, cat, primate, chicken, sheep, goats, llamas, dromedaries, yaks, elks and moose.

The samples of the present invention are typically derived from breeding animals.

The methods of the present invention determine analyte(s) in samples.

An “analyte” or substance or component which presence in a sample is of interest in the present invention, in particular in the field of agriculture and animal breeding, is preferably selected from:
steroid hormones, such as progesterone, estrogens, testosterone,
marker for pregnancy, like proteins and/or hormones, such as pregnancy specific protein B (PSPB), prolactine,
endotoxins, such as lipopolysaccharids (LPS) and glucans, such as β(1-3)glucan,
marker compounds for metabolism disorders, like ketosis, such as β-hydroxy butyric acid or salts thereof.

Further analytes are known to the skilled artisan. Thus, the result derived with the methods, test kits and devices of the invention are important for the farmer and agricultural personnel etc. in particular for reproduction management purposes.

The “container(s)” for the methods, test kits and devices of the present invention are preferably made of transparent and/or single-coloured material, preferably white material.

Preferably the container material is transparent, additionally or alternatively monochromic, particularly white. For instance a white container provides a good effect of contrast towards a precipitate, which is formed by the reaction of the precipitating enzyme substrate, respectively the precipitating chromogen with the enzyme-labelled conjugate/antibody (see below).

The container is preferably a well of a microtiter plate, a well of a microtiter plate stripe, a mini or micro channel, a hollow fibre, a nano channel, a vial or a micro vial. Further containers are known to the skilled artisan in the field of micro- and nanotechnology.

The container has an inner surface and an inner volume, wherein the inner surface can be coated with antibodies and the inner volume can receive/hold liquids and reagents.

The methods of the present invention can be performed in different preferred formats, which are explained in the following:

Competitive Method Format (Steps a to d)

In a step (a), at least one container is provided, wherein the inner surface of said container is at least partially coated with an anti-analyte antibody.

The term “antibody” in the present invention refers to monoclonal antibody, polyclonal antibody, genetically-engineered antibody, but also to fragments and portions thereof which are still capable of binding to the analyte. Such antibody fragments and portions include Fv, scFv, Fc, Fab etc. Genetically-engineered antibodies include diabodies, chimeric antibodies, humanized antibodies etc

Methods for producing anti-analyte antibodie(s) are well known in the art.

In a subsequent step (b), the sample and an analyte-enzyme conjugate are added to the at least one container.

The analyte of the sample and the analyte-enzyme conjugate will compete for the binding sites of the anti-analyte antibody which is coated at the inner surface of the container.

Preferably, the enzyme of the analyte-enzyme conjugate is selected from peroxidase, such as HRP, or alkaline phosphatase.

Preferably, the analyte-enzyme conjugate comprises the entire analyte or fragments or portions of the analyte, such as peptide(s) for a protein analyte. The skilled artisan is able to select suitable fragments or portions of a desired analyte. See also Example 4. The suitable fragments or portions of a desired analyte are recognized by the anti-analyte antibody used in the invention, i.e. they bind to them.

Methods for producing suitable conjugates are known in the art. See also Greg T. Hermanson, Bioconjugate Techniques. Academic Press, 1996.

Preferably, the volume ratio or quantity between sample and analyte-enzyme conjugate is less than 1, preferably 1 to 3.

In a preferred embodiment, 1 drop of sample (such as raw milk) and up to 3 drops of conjugate solution are used.

However, the volume ratio can also be 1, such as the same number of drops.

Preferred is a ratio of quantity between the sample added and the analyte-enzyme conjugate added, which is smaller than one. Especially preferred is the ratio smaller or equal 1:2, preferred 1:3 and at least 1:10. For instance one drop of a sample and three drops of a conjugate can be added. Particularly using the inventive procedure, the sample is added undiluted and without any pre-treatment.

The sample and the analyte-enzyme conjugate can be added at the same time or consecutively. They can be premixed. Preferably, the sample to be analyzed and the analyte-enzyme conjugate are added simultaneously or basically simultaneously, premixed or subsequently. This way the method offers a flexible addition of a sample and of the conjugate.

In a subsequent step (c), the sample and the analyte-enzyme conjugate are incubated in the at least one container.

Preferably, the incubating is less than 10 minutes, preferably less than 8 minutes, more preferably in the range of 6 and 1 minute.

Preferably, the predetermined incubation time of the sample to be analyzed and of the conjugate is shorter than 10 minutes or shorter than 8 minutes. The predetermined incubation time is preferably at least 10 seconds.

In a subsequent step (d), the mixture of sample and analyte-enzyme conjugate are removed from the at least one container or the container is emptied.

The binding sites of the anti-analyte antibodies are now occupied with the analyte of the sample and/or the enzyme-labelled analyte-conjugate.

Sandwich Method Format (Steps a to d)

In a step (a), at least one container is provided, wherein the inner surface of said container is at least partially coated with an anti-analyte antibody.

In this method format the anti-analyte antibody is also referred to as the primary antibody.

In a subsequent step (b), the sample is added to the at least one container.

In a subsequent step (c), the sample is incubated in the at least one container. After the incubation the sample is removed from the at least one container or the container is emptied.

In a subsequent step (d), a secondary antibody conjugated with enzyme is added to the at least one container, the secondary antibody is incubated in the at least one container. After the incubation the secondary antibody is removed from the at least one container or the container is emptied.

Preferably, the enzyme of the secondary antibody is selected from peroxidase, such as HRP, or alkaline phosphatase. Such secondary antibodies that are conjugated with enzymes are known in the art and also commercially available. Preferably, the secondary antibody is specific for the primary antibody, i.e. the anti-analyte antibody.

Preferably, the incubating in step (c) and/or (d) is less than 10 minutes, preferably less than 8 minutes, more preferably in the range of 6 and 1 minute.

Preferably, the predetermined incubation times in step (c) and/or (d) are shorter than 10 minutes or shorter than 8 minutes. The predetermined incubation times are preferably at least 10 seconds.

Steps e to g

In a step (e) of the methods of the invention, a precipitating enzyme substrate is added to the at least one container.

Preferably, the precipitating enzyme substrate comprises or contains a precipitating chromogen.

The precipitating enzyme substrate is selected dependent on the enzyme used in the conjugate or secondary antibody.

For peroxidase, the enzyme substrate is preferably selected from
TMB (3,3′,5,5′-tetramethylbenzidine),
DAB (3,3-diaminobenzidine),
4-CN (4-chloro-1-naphthol) and
AEC (3-amino-9-ethyl carbazole).

For alkaline phosphatase, the enzyme substrate is preferably selected from
BCIP (5-bromo-4-chloro-3-indoxyl phosphate),
NBT (Nitro Blue Tetrazolium chloride) and
INT (iodonitro tetrazolium chloride).

The enzyme of the conjugate or the secondary antibody reacts with the enzyme substrate and a precipitate is formed or precipitation occurs, respectively.

Preferably, after the incubation the precipitating enzyme substrate is removed from the at least one container, wherein the precipitate formed remains at least partially in the at least one container.

Preferably, the precipitating enzyme substrate solution/chromogenic mixture respectively the excess substrate is removed out of the at least one container after a predetermined incubation time, whereas a formed precipitate at least partially remains in the container. Particularly the precipitate adsorbs at least partially at the inner surfaces of the container and results for example in a colour change at the inner surface of the container, which serves as a measure for the analyte content of the sample analyzed in the way described herein.

By removing of the enzyme substrate the reaction of the substrate with the bound enzyme-labelled conjugate or secondary antibody is terminated.

Preferably, the precipitating enzyme substrate is incubated for less than 5 minutes, preferably less then 3 minutes, more preferably in the range of 2 minutes and 30 seconds.

Preferably, the predetermined incubation time of the precipitating enzyme substrate is shorter than 5 minutes or shorter than 3 minutes, The predetermined incubation time is preferably at least 10 seconds.

Importantly, as a result of the short incubation times for sample, conjugate(s) and enzyme substrate respectively, the inventive procedure offers a rapid test for the determination of the analyte content of a sample.

In a subsequent step (f), the colour value of the inner surface of the at least one container is detected.

The “colour value” means the shade of colour, the colour saturation, the white shade value and/or the grey scale level.

The colour value changes according to the precipitate which is formed or is being formed. The colour value depends on the amount of precipitate formed.

Due to the reaction of the precipitating enzyme substrate with the enzyme of the conjugate or the secondary antibody or due to the (chromogenic) precipitate formed thereby, the colour value at the inner side of the at least one container changes and is, therefore, a measure for the analyte content of the sample to be analyzed.

Since in the competitive embodiment of the method according to the present invention, the analyte of the sample to be analyzed and the enzyme-labelled analyte of the conjugate compete for the binding sites of the anti-analyte antibody coating, a high detected colour value, for instance a high colour saturation value, of the container corresponds to a low analyte content of the analyzed sample. Vice versa, a low detected value, for instance a low colour saturation or a high white shade value (when a white container is used), respectively, corresponds to a high analyte content of the analyzed sample.

In the sandwich embodiment of the method according to the present invention, the colour value correlates with the analyte content of the sample, i.e. is directly proportional.

In an optional subsequent step (g), the colour value detected in step (f) is converted into a value of the analyte content of the sample.

This is preferably performed by using a calibration curve which was generated before by linear regression using the detected colour value of one or more standards and internal calibration values, as described below.

Preferably, a lot-dependent calibration value is used for the conversion of the value of the analyte content of the sample in step (g).

In a preferred embodiment, a lot-dependent calibration value is accounted for the conversion or calculation or translation.

Thereby, “lot” or “batch” means, that the reagents used in one lot or batch exhibit the same properties, e.g. because they have been produced in one continuous production process. That means, a measurement of a sample using reagents of one lot or batch produces (under identical external conditions) the same result of measurement. Thus, a calibration value for an entire lot or batch can be determined via a single calibration measurement.

Thus, in the method of the present invention, the reactivity respectively the activity of the various reagents of one lot, respectively the antibody coating and the conjugate, can be respected in the conversion of the value detected in a value for the analyte content by considering the lot-dependent calibration value.

Preferably, step (f) is performed concomitantly with step (e), in an overlapping manner with step (e) or at defined point(s) of time during step (e), preferably in a continuous, temporary and/or punctual manner.

This means, the detection can be performed after and additionally or alternatively during the reaction of the precipitating enzyme substrate respectively the precipitating chromogen with the enzyme of the analyte conjugate.

Preferably, the colour value, e.g. the grey scale value in the container is acquired continuously, temporarily and/or punctually during the reaction. By this means e.g. the course of the changing of the value measured, e.g. the grey scale value, can be recorded during the reaction. For instance to determine an appropriate point in time for aborting the reaction or the optimal measurement.

In a preferred embodiment, the methods of the invention further comprise the step of measuring a base line colour value by detecting the colour value of the inner surface of the at least one container before step (b), wherein the base line colour value is preferably used for the calibration of the colour value detected in step (f).

The “base line” or “zero reference” refers to the colour value of the container before sample is added, but anti-analyte antibody is already coated.

Thus, the colour value of the inner surface of the at least one container is recorded as a base line or zero-reference-determination before the addition of a sample to be analyzed. A base line or zero value determined subsequently can serve for the calibration of the value determined. Especially the value of the zero-reference-determination of this container is subtracted from the value determined for this container or the difference is calculated respectively. By the use of this calibration or zero-point-determination a falsification of the results measured respectively the value determined for this container is prevented, for instance impurities which might be present in the container before addition of the sample or colour variations of the material of the container are considered.

In a further preferred embodiment, the methods of the invention further comprise the utilization at least one standard analyte solution which is measured in parallel to the sample, wherein the standard analyte solution has a known analyte content.

The standard solution is treated/utilized in the same way as the sample in the steps of the methods of the invention at the same time as the sample but in different containers.

Thus, all conditions and treatments which are described for the sample also apply for the standard, i.e. incubation times, wash steps etc.

A known analyte content can be zero (i.e. contain no analyte) or a given concentration. Several standard solutions can be used, preferably a zero standard and a standard with a given concentration.

Using at least one second container, a standard determination using a standard solution with a defined analyte content is provided. Alternatively, at least two containers are provided, which comprise or are filled with different standards. These standard measurements serve as function check of the anti-analyte antibody coating and of the reagents used respectively for the proper operation of the test procedure in general. This means, that thus a functional incapability or a reduced functional capability of the procedure e.g. caused by the ageing of the reagents can be considered. Alternatively or additionally, the standard determination also covers the influence of extern variables such as the influence of the ambient temperature or the air humidity on the determination of the analyte content.

Preferably, the standard solution or one of the standards is a zero standard, meaning that the solution is free of analyte. A further standard solution has a given analyte content which is unequal zero. When using a zero standard, the competitive embodiment of the method of the invention results in a maximum detected colour value (e.g. in a maximum grey scale value or a minimum white shade value). If the standard determination does not result in a maximum colour value or respectively not in a change of the colour value in the container, it can be concluded, that one of the reagents or the coating is not functional or there is an application error, e.g. a reagent has not been applied. Additionally, a detected colour value of an analyzed sample can be calibrated by the detected maximum colour value of the standard determination. For instance, the maximum created/generated colour value by the zero standard can be used for the definition of a maximum producible measurement with the reagents used under the existing external conditions (e.g. ambient temperature, air humidity).

In a further preferred embodiment, the methods of the invention further comprise wash step(s) with a wash solution.

Preferably, after removing of a solution from a container (such as mixture of standard solution/sample and conjugate or sample, secondary antibody) washing step(s) are performed using a washing solution, especially a washing buffer solution. By this means unbound reagents are removed from the container respectively from the inner surface of the container and in the container essentially only the bound compounds, such as the analyte bound to the coating, remains.

In a further embodiment of the methods of the invention, the inner surface of the container is at least partially coated with a further antibody.

Such a further antibody can be specific for the anti-analyte antibody and can serve as a spacer between the inner surface and the anti-analyte antibody or help to orient the anti-analyte antibody for optimal binding with the analyte as well as to allow an efficient coating with anti-analyte antibody. Such a further antibody is also called a capture antibody.

The further antibodies arrange themselves on the inner surface of the container in any orientation, this means at least a part arranges themselves in a way, that the binding sites of these further antibodies are orientated to the inner side of the container. By providing of the anti-analyte antibody coating the anti-analyte antibodies attach only to those of the further antibodies, whose binding sites are orientated to the free volume of the container. An optimal orientation of the anti-analyte antibodies, i.e. their binding sites are orientated to the inner side of the container, is assured this way. This way, almost all anti-analyte antibodies of the coating provide binding sites for the analyte of a sample added as well as an analyte-enzyme conjugate.

Such a further antibody can also be a different anti-analyte antibody:
for the same analyte but with a different binding site (for increasing sensitivity)
for a further analyte to allow the detection of more than one analyte.

The measurement range of the methods of the present invention is between 0.1-100 ng analyte per ml sample, preferably 0.5-10 ng/ml, more preferably 0.5-5 ng/ml.

For instance, by testing and adjusting different dilutions of anti-analyte antibodies in the production process of the coated containers and by testing and adjusting different dilutions of sample and conjugate/secondary antibody the measurement range can be optimized and then adapted for specific needs.

Test Kit

As outlined above, the present invention provides a test kit for the determination of the analyte content in a sample.

The test kits of the invention are preferably for performing the methods of the invention and/or for use in the device of the invention.

The test kits comprises the following components:

(i) a device with a plurality of containers as defined herein, wherein the inner surfaces of the plurality of containers is at least partially coated with an anti-analyte antibody,

(ii) at least an analyte-enzyme conjugate or anti-analyte antibody conjugated with enzyme, as defined herein,

(iii) at least a precipitating enzyme substrate (or a solution thereof), as defined herein,

(iv) at least an standard analyte solution,

(v) optionally, a wash solution.

The at least an standard analyte solution (iv) has a known analyte content.

The test kit device (i) with a plurality of containers is preferably a test plate having a number of containers, preferably membrane free containers for the acceptance of samples to be analyzed, whereas the inner surfaces of the containers are at least partially coated with anti-analyte antibodies.

Preferably the container material is transparent, additionally or alternatively monochromic, particularly white. The container is preferably a well of a microtiter plate, a well of a microtiter plate stripe, a mini or micro channel, a hollow fibre, a nano channel, a vial or a micro vial. Thus, the device (i) is a microtiter plate, a microtiter plate stripe etc.

More preferred, the device (i) is a microtiter plate, especially a test plate revealing a number of microtiter plate strips or individual cavities (containers).

Preferably at least one of the number/plurality of containers of the test kit device (i) is used as “standard container” for the standard for a control determination. More preferably, only one out of the containers of the a test plate (device (i)) or only one container of each of one test strip (device (i)). For instance the standard is a zero standard and results in a maximum value of determination as described herein before (for the competitive format).

Preferably the functional capability and additionally or alternatively the influence of extern variables such as temperature or air humidity on a measurement, respectively on a determination is evaluated using the standard, as described herein before.

The test kits further comprise preferably a calibration code, a calibration value and/or a calibration curve for the lot-dependent calibration.

That means, the calibration code of the test kit accounts for/factors e.g. the reactivity of the reagents used for the respective lot or batch (e.g. the antibody coating), and thus can be used for the conversion of a detected colour value into the actual value of the analyte content. That means, for each lot or batch, whereas a lot can comprise several test kits.

The manufacturer can perform only one standard determination in order to generate one calibration value for all test kits of a lot. Also, the manufacturer can perform several or multiple standard measurements within a lot, wherein the respective average value is used for the determination of the calibration value or the calibration code for test kits of this specific lot.

Preferably the calibration code respectively the calibration curve comprises a calibration over the entire measurement range of the test kit. Thus, the test kit features a calibration for each value detected of a sample analyzed.

Preferably, the test kit contains a washing solution, especially a washing buffer solution, for instance to wash out conjugate mixtures and excess material after removing the sample from the number of containers, so that basically the antibody coating and the analyte of a sample or of the conjugate bound to the antibody coating in a specific ratio to each other remains in the containers.

Device

As outlined above, the present invention provides a device for the determination of the analyte content in a sample.

The devices of the invention are preferably for performing the methods of the invention and/or for the use with the test kits of the invention.

There are several preferred embodiments of the devices of the invention,

In one embodiment, a device of the invention consists of components (a) to (d), which are described below.

In a further embodiment, a device of the invention consists of components (a), (b), (d) and (e), which are described below.

In a further embodiment, a device of the invention consists of components (a) to (e), which are described below.

Component (a) of the device of the invention is a receptacle (16) for at least one container (2), wherein the at least one container is preferably as defined herein.

Component (b) of the device of the invention is at least one dosing unit (18) for dispensing a given amount of reagents and/or sample(s) (6) to the at least one container (2).

Component (c) of the device of the invention is at least one evacuation unit (20, 21) for removing, in particular evacuating, of reagents and/or sample(s) (6) from the at least one container (2).

Component (d) of the device of the invention is at least one sensor (22) for generating a sensor signal as measurement for the colour value of the inner surface of the at least one container (2).

The colour value is the shade of colour, the colour saturation, the white shade value and/or the grey scale level (as described herein).

Component (e) of the device of the invention is a control/evaluation unit (30).

The control/evaluation unit (30) comprises an input unit or input interface (32). The input unit or input interface (32) serves for entering at least one calibration code, calibration value and/or calibration curve, wherein the control/evaluation unit (30) generates or calculates a value of the analyte content in the sample from the sensor signal detected by using the at least one calibration code, calibration value and/or calibration curve.

Preferably, the device has a control/evaluation unit, which preferably has an input interface to enter at least one individual calibration code or -value and/or an individual calibration curve. Thereby, via the control/evaluation unit and using said calibration values, that have been entered, a value of the analyte/substance content can be generated or calculated from the at least one sensor signal detected. Wherein the term “generated” or “generable” means in this context e.g. the transformation of a sensor signal detected, e.g. a grey scale value in an actual value of a substance content respectively a substance concentration of a sample analyzed.

Preferred is a device, wherein one element of the evacuation unit (20, 21), in particular the evacuation line (20), is arranged at the dosing unit (18).

Preferably, an evacuation element, especially an evacuation line or trunk is arranged to the dosage unit. By means of the evacuation unit and its element(s), a solution, respectively sample or reagent, present in the at least one container, can be evacuated, preferably into a waste container.

Preferably, the evacuation unit (20, 21) is connected to a drive unit or arranged with it, so that the evacuation unit (20, 21) via control of the control/evaluation unit (30) can be inserted into a container (e.g. a sample container), for instance by kneeling the dosing unit and/or the evacuation unit.

In a preferred device, a lot-dependent calibration code or value is or can be entered into the input unit or input surface (32) of the device.

Preferably, a lot dependent calibration code or -value, especially a calibration code as previously described in context with the test kits/methods, can be entered via the input-interface.

In a preferred device, the receptacle (16) is designed for at least two containers (2), wherein at least one container is a sample container and at least one container is a standard container.

Preferably, the receptacle (16) can be loaded with at least two containers, whereas one of the containers is standard container for a standard determination and at least one of the containers is a sample container. By means of the control/evaluation unit (30) the value of at least one standard determination, especially of only one single standard determination using at least one standard solution revealing a given substance amount for calibration purposes, preferably for functional control of used reagents or for consideration of extern variables on the determination of the substance content using the device.

In a preferred device, the at least one sensor (22) is a contrast sensor, in particular a white light contrast sensor, or a photometer.

Preferably, the at least one sensor (22) is a contrast sensor, in particular a white light contrast sensor, whereas preferably up to 1000 grey scale levels can be detected. Alternatively, the at least one sensor is a photometer, which measures transmitted light through the container for instance a colour change. In this case the application of transparent recipients is preferred.

In a preferred device, prior to adding sample(s) the at least one sensor (22) is designed to detect at least one sensor signal of the inner surface of the at least one container as base line value of the container, wherein the control/evaluation unit (30) is preferably designed to use the base line value for calibration.

Preferably, the at least one sensor signal of the inner surface is detectable as zero point or base line determination for the inner surface of the container using the at least one sensor (22) before addition of a sample to be analyzed, whereas via the evaluation/control unit (30) the zero point/base line determination for the calibration of the at least one detected sensor signal of the container is considered, especially in regard to the procedure and test kit described.

In a preferred device, the value of at least one standard measurement, preferably one standard measurement, with at least one standard analyte solution is factored by the control/evaluation unit (30) for calibration.

In a preferred device, the at least one sensor (22) detects the at least one sensor signal before and/or during the reaction, preferably in a continuous, temporary and/or punctual manner during the reaction.

The “reaction” refers to e.g. the reaction of a (precipitating) enzyme substrate with the enzyme of the conjugate or secondary antibody, as described herein.

Preferably, by using the at least one sensor (22), the at least one sensor signal is detectable after, and additionally or alternatively during the reaction, especially continuously, temporarily and/or punctually during the reaction. Due to the continuous registration of the sensor signal a time response of a reaction (i.e. the progress of the reaction over time) can be registered and monitored in the at least one container.

A preferred device further comprises a temperature sensor.

Preferably, the device has a temperature sensor for the detection of the ambient temperature. For instance by detection of a too high or a too low temperature for the desired reaction, an evaluation procedure of the device can be aborted, as otherwise possible falsified respectively unusable values would be determined.

A preferred device further comprises a heating unit and/or a cooling unit, preferably for controlling the temperature in the at least one container (2).

Preferably, the device has at least one heating unit, which controls the temperature of the receptacle (16) for the at least one container and/or the at least two containers. Using the heating unit an optimal temperature range for a reaction running in the containers can be generated. Alternatively or additionally, the device has a cooling unit, in order to also generate an optimum temperature for a reaction in the containers, e.g. if the ambient temperatures are too high.

For a further description of the device, in particular for performing the methods and/or using the test kits of the present invention, see below.

The following drawings and preferred embodiments and examples illustrate the present invention without, however, limiting the same thereto.

### BRIEF DESCRIPTION OF THE DRAWINGS

The drawings show:

FIG. 1A the cross section of a container which inner surface is coated with anti-analyte antibody;

FIG. 1B-1F individual procedural steps of the detection method of the invention

FIG. 2 the local section of a device for the determination of the analyte content of a sample (compare to DE 101 19 696 B4), and

FIG. 2A a block diagram of the basic units of the dosing device.

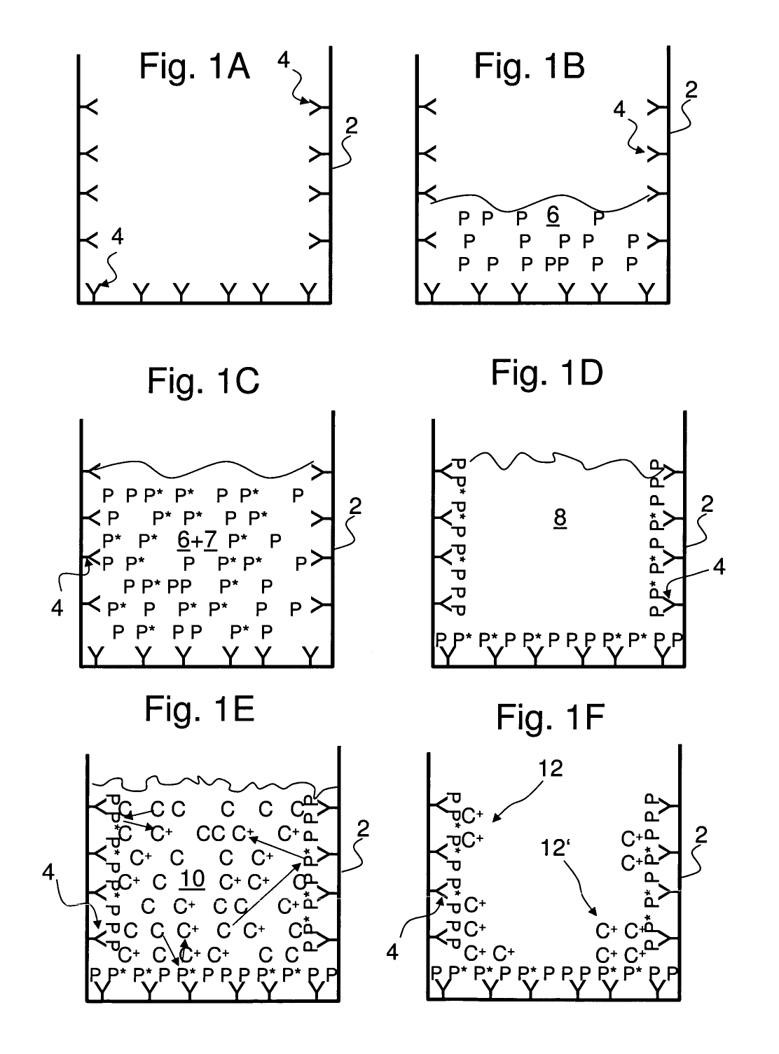

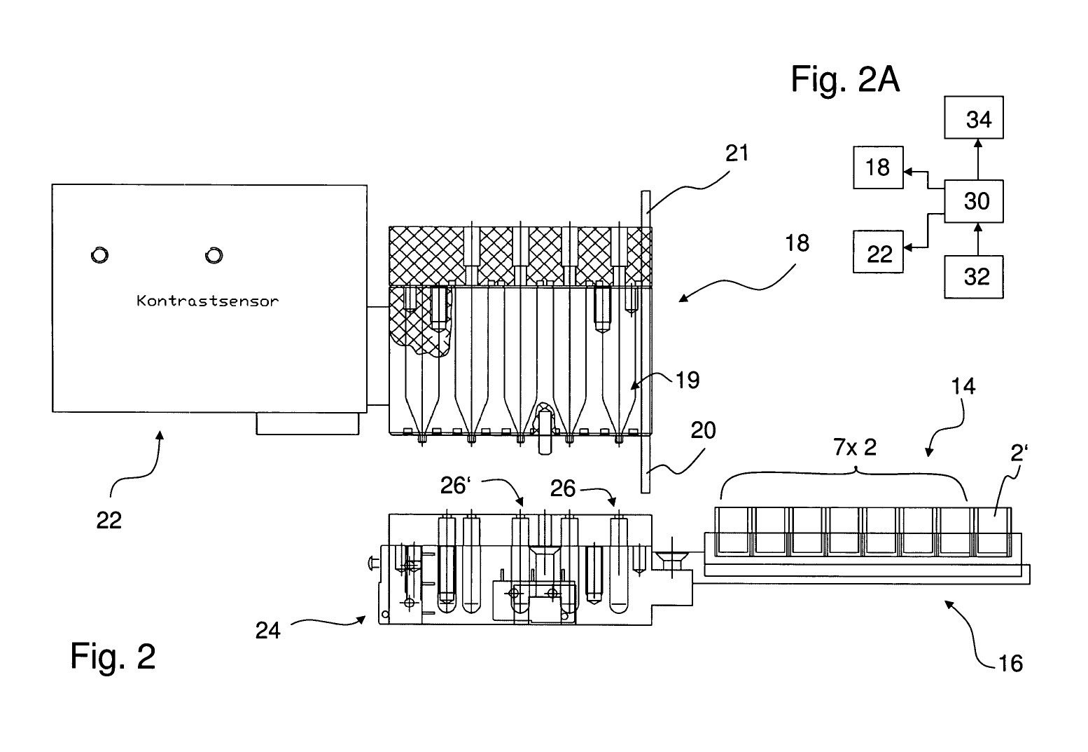

The FIGS. 1A to 1F show the steps of the method for the determination of the analyte content of samples, for instance milk. The method can be applied using the test kit of the invention (not shown) and using the device of the invention (FIG. 2).

FIG. 1A shows the cross section of a container 2, for instance the well/cavity of a microtiter plate or of a microtiter plate strip, which is coated with anti-analyte antibodies (4). Preferably, before providing the anti-analyte antibody-coating on the inner surface of the container 2 a further antibody (not shown) is applied on the inner surface of the container 2. As the subsequently applied anti-analyte antibodies 4 only bind to free binding sites (directed to the inner volume of the container) of the further, initially applied antibody, it is assured, that almost all anti-analyte antibodies 4 added to the container arrange themselves during the coating process in a way, that their binding sites are oriented to the inner volume of the container. When performing the anti-analytic antibody coating, in this way the range of variation of the provided binding sites is reduced as well as the reproducibility of the test is increased.

FIG. 1B shows the container of FIG. 1A after addition of sample 6 (e.g. raw milk), which contains a certain amount of analyte P, e.g. progesterone.

FIG. 1C shows a mixture of the already added sample 6 (e.g. raw milk) and additionally added analyte-enzyme conjugate 7 (e.g. progesterone-enzyme conjugate) in the container 2, whereas the enzyme-labelled analyte (e.g. progesterone) of the conjugate is marked with P*. Preferred is a quantity ratio of sample 6 to conjugate of 1:3. For instance, one drop of sample (milk) 6 and three drops of conjugate 7 are added to the container 2. The analyte (progesterone) P of the sample (milk) 6 and the enzyme-labelled analyte (enzyme-labelled progesterone) P* of the conjugate compete for the binding sites of the anti-analyte antibody 4 (e.g. anti-progesterone antibody) at the inner surface of the container 2. After a predetermined interacting time respectively incubation time of the mixture of sample and conjugate in the container 2, this mixture is removed from the container 2.

In FIG. 1D a wash step is shown, whereas via a washing solution 8 unbound reagents, i.e. sample (milk) 6 and conjugate 7 are washed out. As demonstrated in FIG. 1D, after performing the wash step only the bound analyte (progesterone) P of the sample (milk) as well as the enzyme-labelled analyte (progesterone) P* of the conjugate are bound to the antibodies 4. The ratio between the analyte of the sample (e.g. “milk progesterone”) P and the enzyme-labelled analyte (progesterone) P*, which are bound to the anti-analyte (progesterone) antibody coating, corresponds to the initial concentration ratio between the analyte of the sample (progesterone of the milk) 6 to the conjugate, whereas the analyte (progesterone) concentration of the conjugate is known. This way, the initial analyte (progesterone) content P of the sample can be concluded from the ratio of the bound sample analyte (milk progesterone) P to the analyte (progesterone) P* of the conjugate, respectively it represents the measure therefore.

In FIG. 1E, after removal of the wash solution 8, a precipitating enzyme substrate solution 10 with a precipitating chromogen is added to the container. The initially colourless component C respectively the chromogen of the enzyme substrate solution 10 is converted into a precipitating coloured component C⁺ by the enzyme of the enzyme-labelled analyte (e.g. progesterone) P* of the conjugate. The enzyme catalyzes the reaction. These coloured components C⁺ form a precipitate 12, i.e. they are being precipitated or deposited and they are not soluble. The amount of the precipitate 12 is inverse proportional to the concentration of the analyte (e.g. progesterone) P respectively the analyte content of the sample (i.e. the progesterone content of the milk). This means, by adding a zero standard (analyte-free, e.g. progesterone-free) instead of sample (e.g. milk) a maximum value respectively a maximum amount of precipitate 12 is formed. The amount of the formed precipitate 12 therefore is a measure for the initial concentration of the analyte P (e.g. progesterone) in the sample (e.g. milk) 6. For instance, by measuring the change of the white shade value or the grey scale value or level inside the container the initial analyte concentration in the sample (e.g. the progesterone concentration of the milk) can be concluded.

Preferably, the reaction of the precipitating enzyme substrate solution 10 with the enzyme of the progesterone-conjugate is stopped by removing the enzyme substrate solution 10 from the container 2. The precipitate 12 formed accumulates at least partially at the inner surface of the container 2 and induces a change of the colour shade, the colour saturation, the white shade value or the grey scale level of the inner surface of the container 2 in comparison to a value, e.g. a white shade value of the container 2 before the addition of the sample (see FIG. 1A). This change, for instance of the white shade value, is a measure for the analyte concentration in the sample (e.g. progesterone concentration of the milk).

The features and reagents described before in reference to FIGS. 1A to 1F are part of a test kit of the present invention (not shown) for the determination of the analyte (e.g. progesterone) content of samples. The test kit thereby comprises a number of the anti-analyte antibody coated containers 2, as described above, and reagents, such as the conjugate 7 and the enzyme substrate solution 10, as described above. At the production of such test kits for each production lot (identical anti-analyte antibodies and reagents) by the manufacturer a standard measurement is performed in order to determine the reactivity respectively the quality of the test. This standard measurement is converted into a calibration code or calibration value or a calibration curve, which is provided to the user by being attached to the test kit.

FIG. 2 shows a device for the determination of the substance content, e.g. the progesterone content, of one or more samples to be analyzed. FIG. 2 is based on the dosing unit which is disclosed in DE 101 19 696 B4 (which is herewith incorporated in its entirety by reference) and presents a further development in respect of the aspiration/evacuation and the analysis. In a receptacle 16 seven sample containers 2 and one standard container 2′ in the form of microtiter plate strips 14 are arranged. Above the container 2, shifted to the left, the dosing unit 18 is shown in local section. The dosing unit 18 has several intake volumes 19, which can take up certain amounts of various reagents by coupling to a coupling block 24 respectively its manifold 26. The manifolds 26 preferably are connected with the respective storage reservoirs for the reagents (not shown). Via a control/evaluation unit (30, FIG. 2A) the dosing unit 18 respectively the container 2 or the receptacle 16, for instance via stepping motors (not shown), can be moved accordingly, so that defined amounts of the reagents can be dispensed into the respective containers, preferably in order to accomplish the method of the present invention described before.

Furthermore, the device preferably has a contrast sensor 22, in order to detect after a reaction or during a reaction a bright/dark value respectively a grey scale value of the containers 2, respectively a respective sensor signal, which is converted by the control/evaluation unit 30. Additionally, the device preferably has an aspiration unit (only partially shown), whose aspiration pipe 21 is arranged at the dosing unit 18, whereas an aspiration trunk/line 20 protrudes in the direction of the containers 2, 2′. Using this aspiration trunk 20 excess reagents or reagents/samples no longer required can be aspirated from the respective containers 2, 2′, preferably into a waste container (not shown) in order to initiate the appropriate disposal of the reagent and sample residues. Via an input interface 32 of the control/evaluation unit 30 various data are entered, e.g. calibration values or information about the origin of the samples analyzed (e.g. milk from cow xy, date, farm e.t.c.).

Subsequently an application of the device for the determination of the analyte progesterone for a number of samples (e.g. milk) by using the test kit described before is exemplified:

Initially, the individual (lot-specific) calibration code of the test kit is entered on the input interface 32 of the device. Thus, a value of a measurement determined later on can be adapted to the lot-specific reactivity respectively quality of the reagents of the test kit. Preferably, the white shade value of each container is read before the addition of samples into the containers 2 (see FIG. 1A), in order to generate a zero standard value (or base line value) for a subsequent contrast measurement. This way, for instance, colour deviations caused by the production technique of the test strips 14 used are considered. Subsequently, milk samples to be analyzed are added to the various containers 2 of the strip 14, whereas one of the containers 2 remains empty for the standard determination. Preferably, one drop of a sample is added to each of the containers 2. Subsequently, the measurement procedure is activated and the dosing unit 18 dispenses the appropriate volumes of the reagents (e.g. conjugate) into the respective containers. By means of the aspiration trunk 20 the containers 2 are emptied respectively their content is aspirated. Directly after the addition of the precipitating enzyme substrate solution 10, the proceeding reaction in the containers can be monitored via the contrast sensor 22. Preferably, the enzyme substrate 10 is aspirated from the containers 2 after a predetermined incubation time in order to terminate the reaction in the containers. In the specifically provided container 2′ for the standard determination, a zero standard is added, so that in this container 2′ a maximum amount of the precipitate 12 is formed. In this manner a simple functional check of the test kit on the one hand and of the device on the other hand is provided, as described before. Additionally, this way using the control/evaluation unit 30, the impact of extern variables (e.g. ambient temperature, humidity) on a measurement can be acquired/considered, as the zero standard (base line value) indicates the maximum possible reading at the conditions present at the measuring time. After removing of the enzyme substrate solution 10 the bright-dark-respectively the grey scale value or the white shade value of each container 2 is determined using the contrast sensor 22 and the respective sensor signal is processed by the control/evaluation unit. By considering the calibration code, the zero point measurement and the standard measurement, a value of a progesterone content of a sample in the respective container 2 is generated respectively calculated by the control/evaluation unit and provided to the user via an output unit respectively via a display 34. Preferably these calculated values can be saved sample specific respectively individually for a subject tested, for instance a cow, in order to register the chronological progression of the progesterone level of this cow.

Examples of the analysis are described further (wherein progesterone is the example analyte)

The ELISA test described herein is based in its basic conception, first of all, on technologies, as described for the progesterone determination for instance by Meyer (1989) (H. Meyer. Enzymimmunologische Meβverfahren zur Hormonanalytik. Enke Verlag 1989). ELISA microtiter plates are coated directly or employing the double antibody method using anti-progesterone antibodies. By performing the test for progesterone determination sample respectively standard solution and progesterone-enzyme conjugate are added. The free progesterone from standard or sample competes with the progesterone-enzyme conjugate for the free binding sites of the antibodies. After a washing step to remove unbound reagents the addition of the chromogen enzyme substrate is performed. The colour reaction, which is inverse proportional to the progesterone concentration, is a measure for the progesterone concentration of the sample analyzed. The method, as described in Meyer (1989), is described with minor modifications also in EP 0 671 006 B1 (1992).

The invention described here describes the conception of an ELISA method, test kit and its application in instruments such as “eProCheck”. The test is preferably based on microtiter plates respectively microtiter plate test strips, which are coated with anti-analyte antibodies, such as anti-progesterone antibodies. The free analyte (e.g. progesterone) of the sample or the standard competes with a constant amount of analyte (progesterone)-enzyme conjugate, in the competitive format of the present invention.

The ELISA method and test kit is designed that it is ideally adapted to the instrument “eProCheck”. Accordingly the test is adjusted such that the measuring range (0.5-10 ng/mL) is achieved in an ideal way, without the need of adding any sample diluting agents. An incubation time of eight minutes only for the sample (standard solution) and conjugate solution (e.g. progesterone-enzyme conjugate) are sufficient for the performance of the test. The enzyme substrate solution (e.g. TMB) as well needs to be incubated only 3 minutes in order to achieve a sufficient colour development. The volumes of the reagents are respectively adjusted such that the dosing of sample respectively standard solutions, of the conjugate solution, of the washing solution and of the substrate solution, regulated by a jet system, as realized for instance in “eProCheck”, can be added.

In principle, the determination of the colour intensity can be carried out in two ways, by using two different measuring cells. In the first option the determination of the bright/dark value of the solution to be determined is performed. In order to avoid negative effects caused by undesired reflexions, in this case not transparent microtiter test strips are used but preferably white coloured test strips. In a further option the determination is carried out in the conventional photometric way.

The complete system of the strip test described here, based on the ELISA technology using a dosing and measurement instrument, such as “eProCheck®” (DE 101 19 696 B4) provide an ideal complete system of analyte analytics, such as for progesterone, on the farm.

### EXAMPLES

### eProCheck System and Instrument

In the following Examples, the eProCheck system (FrimTec GmbH, Germany) is used. See also the description in DE 101 19 696 B4, which is enclosed herein in its entirety by reference. As described earlier eProCheck is an instrument, which performs the dosage of certain volumes of reagents, required for the performance of microtiter plate-based ELISAs and the reflectometric or photometric detection of the colour signal created in the test. Thus, the instrument is ideal for on-site testing on farms or other comparable sites. This way the instrument can run and evaluate ELISA-based tests within several minutes. The aquired sensor signals can be converted into a concentration of target analytes by calculation using internal calibrator values.

The common materials and reagents used are: white coloured (for reflectometric reading) or transparent (for photometric reading) ELISA microtiter plates containing break-apart wells (containers). The containers of the plates (i.e. the wells) are coated with polyclonal antibodies, purified from antiserum. The antibodies are coated in appropriate dilutions in buffer, preferably carbonate buffer pH 9.6, and blocked, such as with analyte-free bovine serum albumin (BSA) in PBS pH 7.4.

The standards are diluted in phosphate-buffered saline (PBS)-based solutions, preferably of neutral pH (˜7.0). The washing buffer (washing buffer or rinsing solution) is also PBS-based and contains an appropriate detergent (such as Tween20 0.1%).

The conjugate can be dissolved in several commercial HRP-conjugate stabilizers. Several commercial available ready-to-use one-step precipitating or non-precipitating TMB-substrate solutions (e.g. Sigma T0440, T0565) are applicable in the tests given.

The analyses described here are based on several reagents, buffer formulations and consumables, which can be specific for just one test or common for all tests described here.

### Example 1

### Determination of Progesterone in Bovine Raw Milk

Description of the entire system, including the dosing:

White-coloured microtiter plate strips are used. The wells of the strips are coated with anti-progesterone antibodies (polyclonal anti-progesterone antibodies, made by immunizing rabbits with KLH-progesterone conjugates according to the state of the art) and blocked according to methods known in the art (see Schneider and Hammock, 1992 J. Agric Food Chem. 40(3):525-530.). The wells of the strips can be used individually in a modular manner. (Lockwell System of NUNC GmbH).

The user fills the well(s) with 3 drops of raw milk (which corresponds to a sample volume of about 90 μL) (rawmilk means foremilk, mainmilk or hindmilk). Two further coated wells are used, to which no sample is added. Then, all wells are started for the test performance with eProCheck.

The eProCheck instrument pipets (doses) the reagents as described below and meets the respective given incubation times:

Addition to the two further wells of 3 drops (about 90 μL) of the standard solutions (in PBS) with the concentrations of 0.5 ng/mL and 10 ng/mL, which will be run in parallel to the sample well(s) for calibration purposes. Immediately thereafter, to each well 80 μL of the diluted progesterone-peroxidase conjugate in a stabilizing solution are added. The conjugates are made by hapten-protein conjugations according to methods of the state of the art (such as described in Greg T. Hermanson, Bioconjugate Techniques. Academic Press, 1996.) and dissolved in appropriate commercial dilution buffers. An incubation time of 8 minutes follows. Subsequently, the wells are each washed twice using a washing buffer solution (170 μL) in eProCheck.

Subsequently, addition of 140 μL of the chromogenic substrate solution (ready to use) to each well. After an incubation time of 3 minutes the colour intensity of the solution or the colour intensity of the precipitate (after removal of the remaining solution) is determined reflectometrically and evaluated using an appropriate software algorithm.

The user receives the information regarding the reproductive status of the cow(s) by means of the progesterone content determined.

### Example 2

### Determination of Progesterone in Porcine Serum

The entire system and the dosing is performed as described in Example 1, wherein porcine serum is applied instead of bovine milk.

### Example 3

### Determination of Progesterone in Bovine Raw Milk

The determination of progesterone in raw milk is carried out as described in Example 1, wherein instead of white coloured strips transparent Break-apart Strips (NUNC Maxisorp) are used and the determination is performed photometrically in the eProCheck system.

### Example 4

### Determination of Pregnancy Specific Protein B in Bovine Raw Milk or Bovine Serum

The entire test system and the dosing of the liquids are performed in close dependence to Example 1.

The test is performed in white coloured microtiter plate strips, which wells are coated with anti-PSPB antibodies (polyclonal antibodies, made by immunizing rabbits with the protein purified from the cotyledons of bovine placenta. Additionally antibodies were made by selecting diverse specific peptide sequences of the protein, wherein these peptides were coupled to appropriate carrier proteins according to methods known the art and then used for subsequent immunization) in an appropriate buffer solution and which are subsequently blocked to prevent unspecific binding with an appropriate blocking solution according to the methods as described in the literature (see Schneider and Hammock, 1992, supra).

The user applies 1-3 drops (corresponding to about 30-90 μL) of raw milk (i.e. foremilk, mainmilk or hindmilk) or diluted serum containing the analyte, to the well(s). Two further coated and blocked wells are provided for the parallel application of standard solutions instead of samples for calibration purposes (standard solutions are added by the instrument). Subsequently the analysis is started by applying the test reagents. The instrument doses the reagents as described here and applies the incubation times given for this specific test.

To the two additionally provided wells, 3 drops (about 90 μl) of the two standard solutions (in PBS) serving as negative and positive controls are added for calibration purposes. Subsequently, to each well 80 μL of a diluted PSPB-peptide-peroxidase conjugate (selected peptides of several sequences present in the protein were coupled to HRP according to methods known in the art) as the competing reagent in a stabilizing solution are added and incubated for 8 minutes. Then, the wells are washed twice using a washing buffer solution (170 μL) applied by the “eProCheck” instrument.

The above can also be carried out with an incubation time of 5 minutes.

Alternatively, in a sandwich assay format the sample (respectively the standard) is applied to the coated wells of the microtiter plate. After a washing step in order to remove unbound analyte (standard), a further anti-PSB antibody conjugated with HRP is applied and incubated for 8 minutes. Then, the wells are washed twice using a washing buffer solution (170 μL) applied by the “eProCheck” instrument.

Subsequently (in both embodiments), to each well 140 μL of a precipitating chromogenic TMB-substrate solution (ready to use) are added. After another incubation time of 3 minutes the liquid is removed and the colour intensity of the surface of the white test strip is determined reflectometrically and evaluated using an appropriate software algorithm.

The above step can also be carried out with an incubation time of 1 minute.

The user receives the information regarding the pregnancy of the cow(s) by means of the protein B content determined in the sample(s).

### Example 5

### Determination of Prolactine in Porcine Serum for Early Pregnancy Detection

The entire test system and the dosing of the liquids are performed in close dependence to Example 1.

The test is performed in transparent microtiter plate strips, which wells are coated accordingly with anti-prolactin antibodies (made by coupling of haptens to proteins and subsequent immunization according to the state of the art) in an appropriate buffer solution and which are subsequently blocked with an appropriate buffer-blockage solution according to methods described in the literature (see Schneider and Hammock, 1992, supra).

The user applies 1-3 drops of appropriate porcine serum (corresponding to about 30-90 μL) to the well(s). Two further coated (and blocked) wells are provided additionally in order to apply standard solutions for calibration purposes (added by the instrument). Subsequently the analysis is started by applying the test reagents. The instrument adds the reagents as subsequently described and applies the incubation times given.

To the two additionally provided wells, 3 drops (about 90 μL) of the two standard solutions (in PBS) serving as negative and positive controls are added, for calibration purposes. Subsequently, to each well 80 μL of a diluted prolactin-peroxidase conjugate (hapten protein+HRP, conjugation according to state of the art) in a stabilizing solution are added and incubated for 8 minutes. Then, the wells are washed twice using a washing buffer solution (170 μL) applied by the “eProCheck” instrument.

The above can also be carried out with an incubation time of 5 minutes.

Subsequently, to each well 140 μL of a chromogenic TMB-substrate solution (ready to use) are added. After another incubation time of 3 minutes the optical density of the substrate solution in the test strips is determined reflectometrically or photometrically and evaluated using an appropriate software algorithm.

The above step can also be carried out with an incubation time of 1 minute.

The user receives the information regarding the pregnancy of the pig(s) by means of the prolactin content determined in the sample(s).

The features disclosed in the foregoing description, in the claims and/or in the accompanying drawings may, both separately and in any combination thereof, be material for realizing the invention in diverse forms thereof.

### LIST OF REFERENCE SIGNS

2, 2′ containers
4 anti-analyte antibody
6 sample, e.g. raw milk
7 conjugate
8 wash solution
10 enzyme substrate solution
12 precipitate
14 microtiter plate strip
16 receptacle
18 dosing/pump unit
19 intake volume
20 aspiration trunk or evacuation line
21 aspiration pipe (evacuation pipe)
22 contrast sensor
24 coupling block
26, 26′ aspiration manifold
30 control/evaluation unit
32 input unit or interface
34 output/display unit
P analyte, e.g. progesterone
P* enzyme-labelled analyte
C colourless component of a chromogenic enzyme substrate
C+ precipitated component of a chromogenic enzyme substrate

1. An immunological method for the determination of an analyte content in a sample, comprising the steps of
(a) providing at least one container, wherein the inner surface of said container is at least partially coated with an anti-analyte antibody,
(b) adding the sample and an analyte-enzyme conjugate to the at least one container,
(c) incubating the sample and the analyte-enzyme conjugate in the at least one container,
(d) removing the mixture of sample and analyte-enzyme conjugate from the at least one container,
(e) adding a precipitating enzyme substrate to the at least one container, wherein the substrate comprises a precipitating chromogen,
(f) detecting the colour value of the inner surface of the at least one container, wherein the colour value is the shade of colour, the colour saturation, the white shade value and/or the grey scale level, and
(g) optionally, converting the colour value detected in step (f) into a value of the analyte content of the sample.

2. An immunological method for the determination of an analyte content in a sample, comprising the steps of
(a) providing at least one container, wherein the inner surface of said container is at least partially coated with a primary anti-analyte antibody,
(b) adding the sample to the at least one container,
(c) incubating the sample in the at least one container and removing it therefrom,
(d) adding a secondary antibody conjugated with enzyme to the at least one container, incubating the secondary antibody in the at least one container and removing it therefrom,
(e) adding a precipitating enzyme substrate to the at least one container, wherein the substrate comprises a precipitating chromogen,
(f) detecting the colour value of the inner surface of the at least one container, wherein the colour value is the shade of colour, the colour saturation, the white shade value and/or the grey scale level, and
(g) optionally, converting the colour value detected in step (f) into a value of the analyte content of the sample.

3. The method of claim 1, wherein a lot-dependent calibration value is used for the conversion of the value of the analyte content of the sample in step (g).

4. The method of claim 1, wherein the sample is an untreated sample, which is selected from raw milk, urine, saliva, whole blood, blood plasma or blood serum and/or which is derived from a mammal or a bird.

5. The method of claim 1, wherein the analyte is selected from
steroid hormones,
markers for pregnancy,
endotoxins, and
marker compounds for metabolism disorders.

6. The method of claim 1, wherein the container is made of transparent and/or single-coloured material, wherein the container is a well of a microtiter plate, a well of a microtiter plate stripe, a mini or micro channel, a hollow fibre, a nano channel, a vial or a micro vial.

7. The method of claim 1, wherein the enzyme of the analyte-enzyme conjugate or of the secondary antibody conjugated with enzyme is selected from peroxidase or alkaline phosphatase, and/or wherein the analyte-enzyme conjugate comprises the entire analyte or a fragment or portion of the analyte.

8. The method of claim 1, wherein the precipitating enzyme substrate is selected from TMB, DAB, 4-CN and AEC for peroxidase or from BCIP, NBT and INT for alkaline phosphatase.

9. The method of claim 1, wherein step (f) is performed concomitantly with step (e), in an overlapping manner with step (e) or at defined point(s) of time during step (e).

10. The method of claim 1, further comprising the step of measuring a base line colour value by detecting the colour value of the inner surface of the at least one container before step (b), wherein the base line colour value is used for the calibration of the colour value detected in step (f).

11. The method of claim 1, further comprising utilizing at least one standard analyte solution which is measured in parallel to the sample, wherein the standard analyte solution has a known analyte content.

12. The method of claim 1, wherein the incubating in step (c) and/or (d) is less than 10 minutes.

13. The method of claim 1, wherein the volume ratio between sample and analyte-enzyme conjugate is less than 1.

14. (canceled)

15. The method of claim 1, further comprising wash step(s) with a wash solution.

16. The method of claim 1, wherein the precipitating enzyme substrate is incubated for less than 5 minutes.

17. The method of claim 16, wherein after the incubation the precipitating enzyme substrate is removed from the at least one container, wherein the precipitate formed remains at least partially in the at least one container.

18. The method of claim 1, wherein the inner surface of the container is at least partially coated with a further antibody.

19. The method of claim 1, wherein the measurement range of the method is between 0.1-100 ng analyte per ml sample.

20. A test kit for the determination of an analyte content in a sample, comprising:
(i) a device with a plurality of containers wherein the containers are made of transparent and/or single-coloured material, wherein the containers are wells of a microliter plate, wells of a microtiter plate stripe, mini or micro channels, hollow fibres, nano channels, vials or micro vials, wherein the inner surfaces of the plurality of containers is at least partially coated with an anti-analyte antibody,
(ii) at least one analyte-enzyme conjugate or anti-analyte antibody conjugated with enzyme,
(iii) at least one precipitating enzyme substrate,
(iv) at least one standard analyte solution, and
(v) optionally, a wash solution.

21. The test kit of claim 20, further comprising a calibration code, a calibration value and/or a calibration curve for lot-dependent calibration.

22. A device for the determination of an analyte content in a sample, wherein the device has components (a) to (d); components (a), (b), (d) and (e); or components (a) to (e),
wherein components (a) to (e) are:
(a) a receptacle for at least one container,
(b) at least one dosing unit for dispensing a given amount of reagents and/or sample(s) to the at least one container,
(c) at least one evacuation unit for removing reagents and/or sample(s) from the at least one container,
(d) at least one sensor for generating a sensor signal as measurement for the colour value of the inner surface of the at least one container, wherein the colour value is the shade of colour, the colour saturation, the white shade value and/or the grey scale level and,
(e) a control/evaluation unit, which comprises an input unit to enter at least one calibration code, calibration value and/or calibration curve, wherein the control/evaluation unit generates or calculates a value of the analyte content in the sample from the sensor signal detected by using the at least one calibration code, calibration value and/or calibration curve.

23. The device of claim 22, wherein an evacuation line is arranged at the dosing unit.

24. The device of claim 22, wherein a lot-dependent calibration code or value is entered into the input unit.

25. The device of claim 22, wherein the receptacle is designed for at least two containers, wherein at least one container is a sample container and at least one container is a standard container.

26. The device of claim 22, wherein the at least one sensor is a contrast sensor or a photometer.

27. The device of claim 22, wherein prior to adding a sample(s) the at least one sensor is designed to detect at least one sensor signal of the inner surface of the at least one container as base line value of the container, wherein the control/evaluation unit is designed to use the base line value for calibration.

28. The device of claim 22, wherein the value of at least one standard measurement, with at least one standard analyte solution is factored by the control/evaluation unit for calibration.

29. (canceled)

30. The device of claim 22, further comprising a temperature sensor.

31. The device of claim 22, further comprising a heating unit and/or a cooling unit for controlling the temperature in the at least one container.

32. The method of claim 2, wherein a lot-dependent calibration value is used for the conversion of the value of the analyte content of the sample in step (g).

33. The method of claim 2, wherein the sample is an untreated sample, which is selected from raw milk, urine, saliva, whole blood, blood plasma or blood serum and/or which is derived from a mammal or a bird.

34. The method of claim 2, wherein the analyte is selected from
steroid hormones,
markers for pregnancy,
endotoxins, and
marker compounds for metabolism disorders.

35. The method of claim 2, wherein the container is made of transparent and/or single-coloured material, wherein the container is a well of a microtiter plate, a well of a microtiter plate stripe, a mini or micro channel, a hollow fibre, a nano channel, a vial or a micro vial.

36. The method of claim 2, wherein the enzyme of the analyte-enzyme conjugate or of the secondary antibody conjugated with enzyme is selected from peroxidase or alkaline phosphatase, and/or wherein the analyte-enzyme conjugate comprises the entire analyte or a fragment or portion of the analyte.

37. The method of claim 2, wherein the precipitating enzyme substrate is selected from TMB, DAB, 4-CN and AEC for peroxidase or from BCIP, NBT and INT for alkaline phosphatase.

38. The method of claim 1, wherein step (f) is performed concomitantly with step (e), in an overlapping manner with step (e) or at defined point(s) of time during step (e).

39. The method of claim 2, further comprising the step of measuring a base line colour value by detecting the colour value of the inner surface of the at least one container before step (b), wherein the base line colour value is used for the calibration of the colour value detected in step (f).

40. The method of claim 2, further comprising utilizing at least one standard analyte solution which is measured in parallel to the sample, wherein the standard analyte solution has a known analyte content.

41. The method of claim 2, wherein the incubating in step (c) and/or (d) is less than 10 minutes.

42. The method of claim 2, wherein the volume ratio between sample and analyte-enzyme conjugate is less than 1.

43. The method of claim. 2, further comprising wash step(s) with a wash solution.

44. The method of claim 2, wherein the precipitating enzyme substrate is incubated for less than 5 minutes.

45. The method of claim 44, wherein after the incubation the precipitating enzyme substrate is removed from the at least one container, wherein the precipitate formed remains at least partially in the at least one container.

46. The method of claim 2, wherein the inner surface of the container is at least partially coated with a further antibody.

47. The method of claim 2, wherein the measurement range of the method is between 0.1-100 ng analyte per ml sample.
